(19) United States
(12) Patent Application Publication
(10) Pub. No.: US 2015/0196518 A1
(43) Pub. Date: Jul. 16, 2015

(54) PHARMACEUTICAL COMPOSITIONS OF MESALAMINE

(71) Applicant: CADILA HEALTHCARE LIMITED, Ahemdabad Gujarat (IN)

(72) Inventors: Brij Khera, Princeton, NJ (US); Sushrut Krishnaji Kulkarni, Ahmedabad (IN); Kiran R. Hothur, Ahmedabad (IN)

(21) Appl. No.: 14/593,067

(22) Filed: Jan. 9, 2015

(30) Foreign Application Priority Data
Jan. 10, 2014 (IN) 97/MUM/2014

(51) Int. Cl. A61K 31/196; A61K 9/48

(52) U.S. Cl. CPC A61K 31/196; A61K 9/4808; A61K 9/4833

(57) ABSTRACT

The present invention relates to pharmaceutical compositions of mesalamine. The composition of the invention is a capsule dosage form filled with a tablet. The invention also relates to process for preparing such compositions. The invention specifically relates to a composition comprising an effective amount of mesalamine having higher bulk density.

### CROSS-REFERENCE TO RELATED APPLICATIONS

This application claims priority under 35 USC 119 of Indian Patent Application No. 97/MUM/2014 filed on Jan. 10, 2014.

### FIELD OF THE INVENTION

The present invention relates to pharmaceutical compositions of mesalamine. The composition of the invention is a capsule dosage form filled with a tablet. The invention also relates to processes for preparing such compositions. The invention specifically relates to a composition comprising an effective amount of mesalamine having higher bulk density.

### BACKGROUND OF THE INVENTION

The active compound aminosalicylic acid (in particular 5-ASA, also known as mesalamine or mesalazine) or its derivatives have been used successfully for a relatively long time for the treatment of intestinal disorders, such as, for example, ulcerative colitis and Crohn's disease.

Ulcerative colitis (UC) is an idiopathic, chronic relapsing and remitting, non-specific inflammatory disease of the colonic mucosa. Acute episodes are characterized by chronic diarrhea, rectal bleeding and abdominal pain. Stool volume correlates directly with disease severity, since the colon becomes increasingly unable to reabsorb water and electrolytes as inflammation of the mucosa increases. Loss of water and electrolytes can lead to dehydration, weight loss and serum electrolyte disturbances. Inflammation of the mucosa leads to erosions, which eventually result in rectal bleeding. Anemia and hypoalbuminemia often develop as the disease progresses. Mucosal inflammation also leads to smooth muscle spasm that, in turn, causes urgency to defecate and tenesmus. Systemic manifestations include anorexia, weight loss, fatigue, fever, increased sedimentation rate, arthritis, eye inflammation, anxiety, tachycardia, and elevation in liver function tests (LFTs).

UC also has a profound emotional and social impact on the affected individual. The etiology and pathogenesis of UC are multifactorial and incompletely understood. One theory is that the disease results from inappropriate activation of the mucosal immune system, resulting in the inflammatory response. Theories regarding the inappropriate activation suggest a role for genetic predisposition and/or environment triggers.

UC is most commonly reported in Northern Europe and the United States; reported less frequently in the Middle East and the Southern Hemisphere; and infrequently seen in South America, Asia and Africa. The annual incidence rate is 10.4 to 12.0 cases per 100,000 people with a prevalence rate of 35 to 100 cases per 100,000 people. Although UC occurs at any age, the incidence peaks at 15 to 25 years and 55 to 65 years. The disease is 30% more predominant in females; and a higher incidence is associated with the Jewish population. The goal of treatment in UC is to induce and maintain remission, and improve quality of life.

Subjects with ulcerative colitis may experience periods of remission (times when the symptoms go away) that can last for months or years. However, most subjects' symptoms eventually return. Active therapy is treatment given to treat UC symptoms when they are active. Maintenance therapy refers to treatment given to subjects to enable them to stay in remission, to maintain their health in a disease-free, or limited-disease, state. Maintenance medications must be taken for a prolonged period of time.

The use of 5-ASA and its derivatives as a chemotherapeutic agent in colonic cancer is likewise known, polyps in the colon and rectum being associated with an increased risk of carcinoma (WO 95/18622).

A coloscopic polypectomy in patients with polyps in the colon and/or rectum results in a considerable reduction in risk of the formation of colonic carcinomas and is recommended as a therapy, in particular in the case of colorectal polyps. The recurrence rate after polypectomy, however, is high and amounts to about 6-30% per year. Aminosalicylic acid is suitable for the longer-term treatment of such patients and lowers the recurrence rate of colorectal polyps.

The action of aminosalicylic acid in the treatment of intestinal disorders, or in the prevention of their recurrence or in the prevention of secondary disorders arising therefrom and possible accompanying disorders, takes place by means of the contact of the active compound directly at the site of the disorder in the intestine. The action of the aminosalicylic acid, or a derivative thereof, is directly related to its local concentration in the intestinal area to be treated.

Since inflammatory processes often affect relatively large sections of the intestinal tract, the pharmaceutical form should spread reproducibly over wide areas of the intestine and release the active compound only at the site of inflammation.

A problem in the treatment with aminosalicylic acid is that the active compound is very easily absorbed and can be excreted via the kidney before its action can occur.

FR 2 692 484 discloses a tablet for the controlled release of 4-ASA in a hydrophilic matrix which consists of swellable polymers forming a gel barrier, and having an enteric coating. After dissolution of the coating, the matrix swells and forms a gel barrier through which the active compound diffuses out. After an approximately two-hour lag phase, the composition disclosed in FR 2 692 484 releases the active compound approximately linearly in the intestine over a period of time of a further 14 hours.

U.S. Pat. Nos. 5,541,170 and 5,541,171 disclose an orally administrable solid pharmaceutical composition for the treatment of ulcerative colitis or Crohn's disease of the colon by selectively administering an effective amount of 5-aminosalicylic acid, or pharmaceutically acceptable salt or ester thereof, to the large intestine, said solid oral dosage form being coated with a layer which is insoluble in gastric juice and in intestinal juice below pH 7, but soluble in colonic intestinal juice, whereby the dosage form releases the 5-aminosalicylic acid, salt or ester to the right side of the colon.

U.S. Pat. No. 6,551,620 discloses an orally administrable pharmaceutical pellet formulation for the treatment of the intestinal tract, which comprises a core and an enteric coating with the core including, as a pharmaceutical active compound, aminosalicylic acid or a pharmaceutical salt or derivative thereof and a non-gel matrix forming polymer.

U.S. Pat. No. 6,893,662 discloses a pharmaceutical composition in a solid unit dosage form for oral administration comprising mesalamine, an inner coating layer with a specific polymer and an outer coating layer with a specific polymer other than that used in the inner coating layer.

US Publication No. 2010/086588 discloses compositions and related methods for treating gastrointestinal disorders, e.g., inflammatory gastrointestinal disorders, irritable bowel disease, gastrointestinal motility disorders, functional gastrointestinal disorders, gastroesophageal reflux disease (GERD), Crohn's disease, ulcerative colitis, and diverticulitis, inflammatory bowel disease, and gastroparesis, with a granulated mesalamine formulation. The publication discloses a granulated mesalamine formulation in the form of a capsule. Also included in the publication are methods to extend remission of ulcerative colitis by administration of a once-daily dosage of granulated mesalamine.

U.S. Pat. No. 6,004,581 discloses an oral modified release composition comprising individually coated granules of mesalamine, each granule comprising: a core comprising 5-aminosalicylic acid (5-ASA) (or a salt or an ester thereof) and a spheronization aid, in particular microcrystalline cellulose, and a coating comprising a semi-permeable polymer, in particular, ethylcellulose. The patent discloses the granules ready to use being essentially spherical as defined by an aspect ratio within 1.00-1.25.

US Publication No. 2007/043004 discloses an oral pharmaceutical formulation in the form of a granulate comprising more than 60%, preferably more than 80%, more preferably more than 90% by weight of mesalamine or a pharmaceutically acceptable salt thereof.

Mesalamine is being marketed in USA in different kinds of dosage forms for a number of years. The delayed release tablets of mesalamine are being marketed as Lialda® by Shire (1.2 gm mesalamine), Asacol® by Warner-Chilcott (400 mg mesalamine) and Asacol HD® by Warner-Chilcott (800 mg). The extended release capsules of mesalamine are available as Apriso® by Salix (375 mg mesalamine), Pentasa® by Shire (250 mg and 500 mg mesalamine) and Delzicol® by Warner-Chilcott (400 mg mesalamine). The dosage forms for rectal administration are also available, such as Canasa®, a rectal suppository by Aptalis Pharma and Rowasa®, enema by Meda Pharms. The capsule dosage form is preferred over the tablet dosage form due to several advantages of the capsule dosage form such as unique mixes and ingredients being possible; sealed hard gelatin capsules can be good oxygen barriers; protection for sensitive ingredients; the shell normally breaks down/opens in 4 minutes; reduced gastrointestinal irritation and odorless, tasteless, easy to swallow, etc.

Pharmaceutical manufacturers in the art have heretofore used mesalamine raw materials to form mesalamine medications without regard to the density of the raw material fractions used. The result of this practice is that the manufacturers have been unable to produce a mesalamine capsule for strengths greater than 500 mg per dosage form. There is still a need to develop an alternate capsule composition for human consumption with such a high dose of mesalamine. The current invention relates to a premix comprising mesalamine and excipients for obtaining higher density material; processing that premix into a dosage form that can be filled into a suitable size capsule. The invention also relates to a preparation for obtaining such a composition.

### SUMMARY OF THE INVENTION

In one general aspect, there is provided a pharmaceutical composition of mesalamine, wherein the composition comprises an outer capsule defining an inner volume; and an inner caplet comprising 600-1200 mg mesalamine; the mesalamine having a bulk density between about 0.3 g/ml and 0.8 g/ml and being compressed to a caplet form dimensioned for fitting within and optimizing the inner volume of said capsule.

In another general aspect of the invention, there is provided a capsule of mesalamine comprising a caplet comprising 600-1200 mg mesalamine, wherein the mesalamine is capable to be compressed into a caplet form dimensioned for fitting within and optimizing the inner volume of said capsule.

In another general aspect of the invention, there is provided a capsule of mesalamine comprising a caplet comprising at least 70% by weight of mesalamine, wherein the mesalamine is capable to be compressed to a caplet form dimensioned for fitting within and optimizing the inner volume of said capsule.

In another general aspect of the invention, there is provided a capsule of mesalamine comprising a caplet dimensioned for fitting within and optimizing the inner volume of said capsule, wherein the total weight of the caplet is at least 900 mg.

In another aspect of the invention, there is provided a capsule of mesalamine comprising a caplet comprising 600-1200 mg mesalamine, wherein the mesalamine is capable of being compressed into a caplet form dimensioned for fitting within and optimizing the inner volume of said capsule and the mesalamine has a bulk density between about 0.3 g/ml and 0.8 g/ml.

In another aspect of the invention, there is provided a capsule of mesalamine wherein the caplet comprises an intimate admixture of mesalamine and one or more pharmaceutically acceptable excipients.

Embodiments of the pharmaceutical composition may include one or more of the following features. For example, the pharmaceutically acceptable excipient may include a diluent, a disintegrant, a binder, a lubricant, a glidant, an antiadherent, a plasticizer, a solvent and the like.

In another aspect of the invention, there is provided a capsule of mesalamine wherein the capsule is a “00” size capsule and the caplet is dimensioned for fitting within and optimizing the inner volume of the “00” size capsule.

In another aspect of the invention, there is provided a premix comprising mesalamine and one or more pharmaceutically acceptable excipients, wherein the premix has a bulk density between about 0.3 g/ml and 0.8 g/ml, preferably between about 0.4 g/ml and 0.7 g/ml.

In still another general aspect, there is provided a process for preparing a pharmaceutical composition wherein the process comprising the steps of: preparing a premix comprising mesalamine and one or more pharmaceutically acceptable excipients so that the premix has a bulk density between 0.3 g/ml and 0.8 g/ml; processing the premix; compressing the processed premix into a caplet; coating the caplet with an enteric polymer; and filling the coated caplet in a capsule.

Embodiments of the pharmaceutical composition may include one or more of the following features. For example, the pharmaceutically acceptable excipient may include a diluent, a disintegrant, a binder, a lubricant, a glidant, an antiadherent, a plasticizer, a solvent and the like.

In another general aspect, there is provided a method of treatment of mildly to moderately active ulcerative colitis and for the maintenance of remission of ulcerative colitis which comprises administering to a human patient in need thereof the pharmaceutical composition of mesalamine as herein described.

The details of one or more embodiments of the invention are set forth in the description below. Other features, objects and advantages of the invention will be apparent from the description.

### DETAILED DESCRIPTION OF THE INVENTION

The inventors of this application have surprisingly found that a higher amount of mesalamine can be filled into suitable size capsules for human consumption by using high bulk density mesalamine. The inventors also invented a premix comprising mesalamine and one or more pharmaceutically acceptable excipients so that the premix has higher bulk density, which can be filled into capsules for human consumption.

The present invention takes advantage of the surprising discovery that, by selecting mesalamine and specific excipients within a particular range of bulk density, it is possible to compress and dimension the premix to form a mesalamine caplet or tablet that is smaller than heretofore formed and is more easily ingested. More particularly, the selection of a particular density range of mesalamine and excipients permits the compression and dimensioning of the premix to form a caplet that optimally fits within and substantially completely fills a capsule.

The term “mesalamine” used throughout the specification refers to not only mesalamine per se, but also its pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs and pharmaceutically acceptable prodrugs thereof. The amount of mesalamine used in the present invention is in the range from about 600 to about 1200 mg in a single or divided dose. A preferred amount of mesalamine used in the composition is about 800 mg.

Important physicochemical characteristics of powders are the density properties such as bulk and tapped density, weight variation and flow properties such as angle of repose. Bulk density is the undisturbed packing density of that substance and tapped bulk density relates to the packing density after tapping a bed of substance until no change in the packing density is seen. Bulk density and tapped density can be determined using compendial bulk density apparatus, such as the method given in Test 616 “Bulk Density and Tapped Density,” United States Pharmacopeia 29, United States Pharmacopeial Convention, Inc., Rockville, Md., 2005 (“USP”).

Bulk density of mesalamine used to prepare the caplet is preferably monitored before production to ensure that the bulk density of the mesalamine is at least about 0.3 g/ml and preferably from about 0.3 g/ml to about 0.8 g/ml, more preferably from about 0.4 g/ml to about 0.7 g/ml. This physical property of the drug is very important for making the caplet/tablet that can be filled into suitable sized capsules for human consumption. In another embodiment, if mesalamine having a bulk density lower than about 0.3 g/ml is used to prepare the composition, the mesalamine is treated with pharmaceutically acceptable excipients to make a premix having higher bulk density and thus ultimately the caplet/tablet can be filled into suitable sized capsules for human consumption.

The composition of the present invention includes a quantity of mesalamine having a specific bulk density. The mesalamine composition is dimensioned to form a caplet for fitting within a capsule in a manner that optimizes the volume of the capsule, i.e., fills the internal volume of the capsule substantially completely. Insertion of the mesalamine composition within the capsule masks the unpleasant and unpalatable taste of the enclosed caplet.

Hard-shell capsules offer a customized dosage form that can be made easily and conveniently in the pharmacy. Size “00” (double zero) is usually the largest capsule size used orally for humans. Hard-shell capsules are generally filled with powder or multiparticulates like granules, pellets, minitablets, microtablets, beads, etc. Hard-shell capsules used in this invention may be made up of gelatin or HPMC. Mesalamine of a proposed bulk density can be compressed to meet the target weight and fit into a “00” size capsule, without an unacceptable incidence of “capping” (i.e., splitting along a plane parallel to the long axis of the capsule) and “picking” (i.e., loss of small punctuate flecks of material from its surface). The capsule size used for the purpose of this invention may be selected from “0el”, “0el+”, “0xel”, “00” or “00el”. The caplet should be dimensioned such that it should fit within the capsule to optimize its inner volume. The caplets may be prepared such that the length should not exceed 22 mm, width should not exceed 7.3 mm and thickness should not exceed 6.6 mm. In other embodiments, the ratio of length to width of the caplet should not be more than 3.1. The capsules may be filled with tablets having different shapes like oval shape, modified capsule shape etc. Tablets with modified caplet shape are preferred.

A “delayed release” composition may be designed to delay the release of the drug for a specified period. Delayed release compositions of the present invention include those that exhibit a delayed-release, e.g., compositions that only begin releasing the drug after a fixed period of time. The delayed release compositions of the present invention may include the compositions which may release substantially no drug within the first two hours after administration and after completion of that the composition may release more than 80% of the drug within next two hours. The composition may release less than about 50%, preferably less than 30%, more preferably less than 10% of total drug within one hour after administration.

As used herein, the term “excipient” means any component admixed with or co-incorporated with the active agent. Excipients may act to facilitate incorporation of the active agent into the substrate, modify the release of the active agent from the substrate, stabilize the active agent, enhance absorption of the active agent, enhance tableting, increase the bulk of the pharmaceutical composition, and the like. Excipients are safe for their intended use at the levels employed in the formulation and are compatible with the active agent. It is within the purview of the present invention to determine the type of excipient to be utilized in combination with the active agent as well as to determine how much excipient is to be added and the objective that the skilled artisan wishes to achieve by adding the same.

The pharmaceutically acceptable excipients may include one or more of a diluent, a disintegrant, a binder, a lubricant, a glidant, an antiadherent, a plasticizer or combinations thereof.

Suitable diluents include, but are not limited to, microcrystalline cellulose, starch, dibasic calcium phosphate, tribasic calcium phosphate, calcium carbonate, dextrose, kaolin, magnesium carbonate, magnesium oxide; sugars such as lactose or sucrose; sugar alcohols such as mannitol, sorbitol or erythritol; and mixtures thereof. The diluent may be added to increase the bulk volume of the powder to facilitate granulation or compression.

Suitable disintegrants include, but are not limited to, croscarmellose sodium, crospovidone, sodium starch glycolate, corn starch, potato starch, maize starch and modified starches, calcium silicates, low substituted hydroxypropyl cellulose. The amount of disintegrating agent is preferably in the range of 5% to 35% by weight of the composition.

Suitable binders include, but are not limited to, hydroxyethyl cellulose, hydroxypropyl cellulose, hydroxypropyl methylcellulose, carbomers, dextrin, ethyl cellulose, methylcellulose, shellac, zein, gelatin, polymethacrylates, polyvinyl pyrrolidone, pregelatinized starch, sodium alginate, gums, synthetic resins and the like.

Suitable lubricants, glidants or anti-adherent agents, include, but are not limited to, talc, metallic stearates such as magnesium stearate, calcium stearate, zinc stearate; colloidal silicon dioxide, finely divided silicon dioxide, stearic acid, hydrogenated vegetable oil, glyceryl palmitostearate, glyceryl monostearate, glyceryl behenate, polyethylene glycols, powdered cellulose, starch, sodium stearyl fumarate, sodium benzoate, mineral oil, magnesium trisilicate, kaolin; and mixtures thereof. It would be appreciated that a person skilled in the art is cognizant of the fact that lubricants, glidants and anti-tacking agents may be used interchangeably. The lubricant, glidant or anti-tacking agent may be present in an amount ranging from 0.1% to 10% by weight of the composition.

Suitable plasticizers include triacetin, diethyl phthalate, tributyl sebecate, polyethylene glycol or mixtures thereof.

The core tablet/caplet may be coated with the delayed release coating. The delayed release property of the dosage form may be achieved by using an enteric polymer. “Enteric polymer” used in the invention may be selected from the group consisting of hydroxypropyl methylcellulose phthalate, cellulose acetate phthalate, cellulose acetate succinate, methylcellulose phthalate, hydroxypropyl methylcellulose phthalate, ethylhydroxycellulose phthalate, polyvinylacetate phthalate, polyvinyl butyrate acetate, vinyl acetate-maleic anhydride copolymer, styrene-maleic mono-ester copolymer, carboxymethyl ethylcellulose, methyl methacrylate-methacrylic acid copolymer (Eudragit L-100 (methacrylic acid copolymer L) or Eudragit S-100 (methacrylic acid copolymer S)), methacrylic acid-ethyl acrylate copolymer (Eudragit L100-55 (dried methacrylic acid copolymer LD) or Eudragit L30D-55 (methacrylic acid copolymer LD)), methacrylic acid-methyl acrylate-methyl methacrylate copolymer (Eudragit FS30D), hydroxypropyl cellulose acetate succinate (HPMCAS), shellac or combinations thereof.

The core tablet/caplet may be coated with a barrier or protective layer. In a further embodiment the barrier layer comprises a non-toxic edible polymer, edible pigment particles, an edible polymer plasticizer, and a surfactant. Materials include, for example and without limitation, materials described in U.S. Pat. No. 4,543,370, incorporated herein by reference. Exemplary barrier and/or protective layers include OPADRY®; OPADRY II® which comprises HPMC, titanium dioxide, plasticizer and other components (e.g. OPADRY® Blue, OPADRY® Clear 03F59016, OPADRY® Green 03B510016); and polyvinyl alcohol-polyethylene glycol copolymer marketed as Kollicoat® IR. Suitable barrier layers, for illustration and not limitation, include Kollicoat® IR (a polyvinyl alcohol-polyethylene glycol graft copolymer) and Kollicoat IR White®.

The thickness of the barrier and/or protective layer can vary over a wide range, but is generally in the range 20 to 3,000 microns, such as on the order of about 25 to 250 microns. Preferably the barrier layer retards the release of mesalamine by less than 5 minutes, preferably less than 4 minutes and more preferably by less than 3 minutes. The barrier and/or protective layer in the pharmaceutical composition may be prepared by a suitable material such that it substantially inhibits the release of the active from their respective compartments in the gastric environment of pH below 4.5.

According to one of the embodiments, a delayed release pharmaceutical composition of mesalamine comprises an enteric coated tablet filled into a capsule, wherein mesalamine has a bulk density between 0.3 g/ml and 0.8 g/ml. In another embodiment, a capsule comprises mesalamine in an amount from 600 mg to 1200 mg and one or more pharmaceutically acceptable excipients. In this embodiment, a premix of mesalamine is compressed into a caplet form dimensioned for fitting within and optimizing the inner volume of the capsule. The caplet comprises an intimate mixture of mesalamine and one or more pharmaceutically acceptable excipients.

In another embodiment, a capsule comprises of a premix of mesalamine, sodium starch glycolate, colloidal silicon dioxide and magnesium stearate. This premix is mixed with microcrystalline cellulose and is granulated with an aqueous solution of polyvinylpyrrolidone. The granules thus formed are lubricated and compressed to form a caplet. In one embodiment, the caplet is coated with an enteric coating layer comprising methacrylic acid copolymer and is filled into a capsule. In another embodiment, the caplet thus formed is seal coated, filled into a capsule and the capsule is coated with an enteric coating layer.

The premix of mesalamine has a bulk density preferably from about 0.3 g/ml to about 0.8 g/ml, more preferably from about 0.4 g/ml to about 0.7 g/ml. The particles of the premix have a mean particle size of less than 250 microns, preferably less than 200 microns, more preferably less than 150 microns and most preferably less than 100 microns.

The pharmaceutical compositions as described herein may be prepared by processes known to the person having ordinary skill in the art of pharmaceutical technology such as direct compression, wet granulation, dry granulation or melt granulation.

In one embodiment, a capsule comprising mesalamine may be prepared by providing mesalamine having a bulk density between 0.3 g/ml and 0.8 g/ml; mixing the mesalamine with one or more pharmaceutically acceptable excipients; compressing the mixture into a tablet/caplet; coating the tablet/caplet with an enteric polymer; and filling the coated caplet in a capsule.

In another embodiment, a caplet of mesalamine may be prepared by mixing and granulating mesalamine having a bulk density between 0.3 g/ml and 0.8 g/ml with one or more pharmaceutically acceptable excipients; drying the granules; lubricating the dried granules; and compressing the granules into a caplet. In another embodiment, a caplet may be prepared by mixing mesalamine having a bulk density between 0.3 g/ml and 0.8 g/ml with one or more pharmaceutically acceptable excipients; lubricating the powder mixture; and compressing the powder into a caplet. In still another embodiment, a caplet may be prepared by mixing mesalamine having a bulk density between 0.3 g/ml and 0.8 g/ml with one or more pharmaceutically acceptable excipients; making slugs of the mixture by dry compression technique; breaking the slugs to provide the granules; lubricating the granules; and compressing the granules into a caplet.

In another embodiment, a caplet may be obtained by compressing coated multiparticulates. For this embodiment, multiparticulates may be in the form of pellets, granules or beads prepared by the techniques known in the art. Pellets may be prepared by extrusion-spheronization process. Granules may be prepared by wet granulation, dry granulation or melt granulation process. Beads may be prepared by coating inert carriers such as sugar spheres or MCC (microcrystalline cellulose) spheres with a solution or dispersion containing the mesalamine. These multiparticulates may be coated with a seal coat/barrier coat/intermediate coat/protective coat and then may be coated with an enteric coat to provide delayed release multiparticulates. The coating layer may comprise some excipients which may provide cushioning effect to the particles which may give protection against breakage of coating during compression.

In another embodiment, a pharmaceutical composition of mesalamine may be prepared by a process wherein the process comprises the steps of: preparing a premix comprising mesalamine and one or more pharmaceutically acceptable excipients so that the premix has a bulk density between 0.3 g/ml and 0.8 g/ml; processing the premix; compressing the processed premix into a caplet; coating the caplet with an enteric polymer; and filling the coated caplet in a capsule. The premix may be prepared by making slugs of mesalamine and one or more pharmaceutically acceptable excipients, and breaking these slugs to obtain granules of proper size and bulk density. Bulk density of mesalamine used in making this premix may be below 0.3 g/ml but the ultimate bulk density of premix is between 0.3 g/ml and 0.8 g/ml.

In still other embodiments, there is provided a method of treatment of mildly to moderately active ulcerative colitis and for the maintenance of remission of ulcerative colitis. The method includes administering to a human patient in need thereof the pharmaceutical composition of mesalamine as per the invention.

In another embodiment, the composition of the present invention comprising mesalamine exhibits bioequivalence to a reference composition of mesalamine. As used herein, a “reference composition” is intended to mean a composition of mesalamine which is currently approved as Asacol HD and which may be used as a reference for a new drug application (NDA) or an abbreviated new drug application (ANDA) under the Federal Food Drug & Cosmetic Act.

The bioequivalence studies were carried out between Asacol HD® tablets (reference) and compositions of the invention (test) in fasted and fed state. The study was monitored in terms of Cₘₐₓ and AUC achieved with the test product and the reference product (Asacol HD®).

In a further embodiment, the present invention provides a pharmaceutical composition comprising mesalamine, wherein the composition comprises an outer capsule defining an inner volume; an inner caplet comprising 600-1200 mg mesalamine; and when administered as two dosage units three times daily for six days provides an in-vivo plasma profile for mesalamine with a mean of Cₘₐₓ ranging from 1 μg/mL to 9 μg/mL, a mean of AUC₀₋ₜ ranging from 6 μg*hr/mL to 34 μg*hr/mL; and a mean of Tₘₐₓ ranging from 10 to 16 hours.

The absorbed mesalamine is rapidly acetylated in the gut mucosal wall and by the liver to N-acetyl-5-aminosalicylic acid (N-Ac-5-ASA) which is excreted mainly by the kidney. In still further embodiments, the present invention provides a pharmaceutical composition comprising mesalamine, wherein the composition comprises an outer capsule defining an inner volume; and an inner caplet comprising 600-1200 mg mesalamine; and when administered as two dosage units three times daily for six days provides an in-vivo plasma profile for active metabolite N-acetyl-5-aminosalicylic acid with a mean of Cₘₐₓ ranging from 2 μg/mL to 7 μg/mL, a mean of AUC₀₋ₜ ranging from 14 μg*hr/mL to 36 μg*hr/mL; and a mean of Tₘₐₓ ranging from 10 to 16 hours.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Example 1

| Sr. | | |
|---|---|---|
| No | Ingredient | % w/w |
| Compaction | | |
| 1 | Mesalamine | 70-80 |
| 2 | Colloidal silicon dioxide | 0.2-2.0 |
| 3 | Magnesium stearate | 0.2-2.0 |
| 4 | Sodium starch glycolate | 1-5 |
| Granulation | | |
| 5 | Microcrystalline Cellulose | 8-15 |
| 6 | Polyvinylpyrrolidone | 1-5 |
| 7 | Purified water | q.s. |
| Lubrication | | |
| 8 | Sodium starch glycolate | 1-5 |
| 9 | Colloidal silicon dioxide | 0.5-2.0 |
| 10 | Talc | 0.5-2.0 |
| 11 | Magnesium stearate | 0.3-2.0 |
| Coating Formulation | | |
| 12 | Methacrylic acid copolymer | 1.0-8.0 |
| | (Eudragit S) | |
| 13 | Acetyl tributyl citrate | 0.1-2.0 |
| 14 | Talc | 0.1-1.0 |
| 15 | Titanium dioxide | 0.1-1.0 |
| 16 | Isopropyl alcohol | q.s. |

Process:

Preparation of a Premix:

Mesalamine, sodium starch glycolate, colloidal silicon dioxide and magnesium stearate were mixed and compacted to provide a premix.

Preparation of a Caplet:

The premix thus prepared was mixed with microcrystalline cellulose and was granulated with aqueous solution of polyvinylpyrrolidone to provide granules of mesalamine. The granules were dried and lubricated. The granules were compressed with proper tooling to provide caplets. The caplets thus formed were coated with an enteric coating dispersion of methacrylic acid copolymer.

Preparation of a Capsule:

The enteric coated caplet was filled in the capsule shell to provide a final capsule dosage form.

### Example 2

| Sr. | | Quantity |
|---|---|---|
| No | Ingredient | (mg/tablet) |
| Granulation | | |
| 1 | Mesalamine | 800 |
| 2 | Microcrystalline cellulose | 100 |
| 3 | Polyvinylpyrrolidone | 21 |
| 4 | Sodium starch glycolate | 36 |
| 5 | Purified water | q.s. |
| Extragranular | | |
| 6 | Sodium starch glycolate | 26 |
| 7 | Colloidal silicon dioxide | 6 |
| 8 | Talc | 5.8 |
| 9 | Magnesium stearate | 5.2 |
| Coating Formulation | | |
| 10 | Methacrylic acid copolymer | 40.80 |
| | (Eudragit S 100 | |
| 11 | Acetyl tributyl citrate | 11.54 |
| 12 | Talc | 7.12 |
| 13 | Titanium dioxide | 1.8 |
| 14 | Isopropyl alcohol | q.s. |
| 15 | Ferric oxide (Red) | 1.10 |

Process:

Preparation of Granules:

Mesalamine, sodium starch glycolate and microcrystalline cellulose were mixed together and the powder mixture was granulated using aqueous solution of polyvinyl pyrrolidone.

Preparation of a Caplet:

The granules were dried and mixed with sodium starch glycolate, talc and colloidal silicon dioxide. The blend was lubricated with magnesium stearate. The lubricated granules were compressed with proper tooling to provide caplets. The caplets thus formed were coated with an enteric coating dispersion of methacrylic acid copolymer.

Preparation of a Capsule:

The enteric coated caplet was filled in the capsule shell to provide a final capsule dosage form.

Mesalamine used to prepare the composition as per Example 2 has a bulk density of 0.483 g/ml and a tapped density 0.694 g/ml. The capsule used to fill the caplet was size “00el”.

We claim:

1. A capsule of mesalamine comprising: a caplet comprising 600-1200 mg mesalamine and one or more pharmaceutically acceptable excipients, wherein the mesalamine is capable to be compressed to a caplet form dimensioned for fitting within and optimizing the inner volume of said capsule.

2. The capsule according to claim 1, wherein the caplet comprises about 800 mg of mesalamine.

3. The capsule according to claim 1, wherein the mesalamine has a bulk density of between about 0.3 g/ml and 0.8 g/ml.

4. The capsule according to claim 1, wherein the caplet comprises an intimate admixture of mesalamine and one or more pharmaceutically acceptable excipients.

5. The capsule according to claim 1, wherein the capsule is a “00” size capsule and the caplet is dimensioned for fitting within and optimizing the inner volume of the “00” size capsule.

6. The capsule according to claim 1, wherein the caplet comprises an enteric coat.

7. The capsule according to claim 1, wherein the capsule does not release any drug for the first two hours of its administration.

8. The capsule according to claim 1, wherein the capsule is coated with an enteric coat.

9. The capsule according to claim 1, wherein the caplet comprises at least 70% by weight of mesalamine.

10. The capsule according to claim 1, wherein the total weight of the caplet is at least 900 mg.

11. The capsule according to claim 1, wherein the caplet comprising a premix comprising mesalamine and one or more pharmaceutically acceptable excipients, wherein the premix has a bulk density between about 0.3 g/ml and 0.8 g/ml.

12. A process for preparing the capsule according to claim 11, wherein the process comprises the steps of: preparing the premix comprising mesalamine and one or more pharmaceutically acceptable excipients so that the premix has a bulk density between 0.3 g/ml and 0.8 g/ml; processing the premix; compressing the processed premix into a caplet; optionally coating the caplet with an enteric polymer; and filling the caplet in a capsule.

13. A method of treatment of mildly to moderately active ulcerative colitis and for the maintenance of remission of ulcerative colitis which comprises administering to a human patient in need thereof the capsule according to claim 1.

14. A pharmaceutical composition of mesalamine comprising an outer capsule defining an inner volume; and an inner caplet comprising 600-1200 mg mesalamine; and when administered as two capsules three times daily for six days provides an in-vivo plasma profile for mesalamine with a mean of Cₘₐₓ ranging from 1 μg/mL to 9 μg/mL, a mean of AUC₀₋ₜ ranging from 6 μg*hr/mL to 34 μg*hr/mL; and a mean of Tₘₐₓ ranging from 10 to 16 hours.

15. A pharmaceutical composition of mesalamine comprising an outer capsule defining an inner volume; and an inner caplet comprising 600-1200 mg mesalamine; and when administered as two capsules three times daily for six days provides an in-vivo plasma profile for the active metabolite N-acetyl-5-aminosalicylic acid with a mean of Cₘₐₓ ranging from 2 μg/mL to 7 μg/mL, a mean of AUC₀₋ₜ ranging from 14 μg*hr/mL to 36 μg*hr/mL; and a mean of Tₘₐₓ ranging from 10 to 16 hours.
